(19) United States
(12) Patent Application Publication
(10) Pub. No.: US 2002/0098248 A1
(43) Pub. Date: Jul. 25, 2002

(54) Method for treating and/or preventing infections caused by methicillin-resistant Staphylococcus aureus

(75) Inventors: Hiroshi Fujiwara, Kokubunji-shi (JP); Ken Sawai, Osaka-shi (JP)

Correspondence Address: Joseph DeBenedictis, BACON & THOMAS, 4th Floor, 625 Slaters Lane, Alexandria, VA, 22314, US

(73) Assignee: Toyo Seiyaku Kabushiki Kaisha, Osaka (JP)

(21) Appl. No.: 09/987,543

(22) Filed: Nov. 15, 2001

(30) Foreign Application Priority Data
Nov. 15, 2000 (JP) 2000-348728

(51) Int. Cl. A61K 35/78

(52) U.S. Cl. 424/725; 424/729

(57) ABSTRACT

This invention relates to a method for treating and/or preventing infections caused by Methicillin-Resistant Staphylococcus aureus in human and non-human animals comprising administering an effective amount of an antimicrobial agent consisting essentially of a soluble extract of tealeaves and/or tea stems of “shan-lin-xi-gao-shan-cha” and/or “bai-hao-wu-long” whose place of origin is Taiwan, wherein the extract is obtained by extraction in hot water.

### BACKGROUND OF THE INVENTION

1. Field of the Invention

This invention relates to a method for treating and/or preventing infections caused by Methicillin-Resistant Staphylococcus aureus (hereinafter, simply referred to as “MRSA”), which belongs to bacteria of genus Staphylococcus which has pathogenecity for human beings, domestic or wild animals and pets. The method of this invention is effective in treating and/or preventing infections caused by MRSA which has been a serious problem as pathogen of nosocomial infections.

2. Description of the Related Art

Staphylococcus are known to cause various inflammations in human beings and animals, such as pyodermatitis, conjunctivitis, sinusitis, otitis, cystitis, pneumonia, myelitis, arthritis, and visceral abscess. Among the variety of bacteria belonging to genus Staphylococcus, Staphylococcus aureus are famous for pathogenic bacteria which cause pyosis in human being and animals. They also produce an enterotoxin which reacts as a pathogen of food poisoning. Recently, MRSA having resistance against many kind of antibiotics, has raised serious problems in case of nosocomial infections or grave enteritis caused by microbial selection.

MRSA, as it stands for, is Staphylococcus aureus which has resistance against methicillin, a kind of antibiotics. MRSA are bacteria which acquired resistance against certain pharmaceutical agents having a methicillin skeleton (e.g., penicillins and cephalosporins) which has an excellent antimicrobial effect against Staphylococcus aureus.

MRSA exhibits drug resistant in a wide range, namely all kind of β-lactam drugs available on the market including all kind of penicillins; all kind of cephem-type agents including first generation, second generation, and third generation thereof, monobactam-type agents; and carbapenem-type agents. Additionally, there has been known that MRSA is resistant against new quinolones. Further, there has been reported novel bacteria which are resistant against vancomycin which has been used to treat infections by MRSA. Thus, there has been an increasing demand for providing an efficacious antimicrobial agent against MRSA.

Furthermore, MRSA is not only resistant against a multitude of kind of antibiotics but also has a tendency to exhibit serious pathogenecity. Particularly, there has been a serious problem that MRSA easily infects to patients with reduced resistance. For example, in the case where patients in an immunodeficient condition due to leukemia or cancer or the like, patients suffering from burn at a high level, bedridden aged persons, or prematurely-born babies in need of intensive care are infected with MRSA, recovery of such patients is difficult.

### SUMMARY OF THE INVENTION

In view of the above, an object of this invention is to provide a method for treating and/or preventing infections caused by MRSA in human and non-human animals.

According to an aspect of this invention, the method comprises administering an effective amount of an antimicrobial agent consisting essentially of a soluble extract of tealeaves and/or tea stems of “shan-lin-xi-gao-shan-cha” and/or “bai-hao-wu-long” whose place of origin is Taiwan, wherein the extract is obtained by extraction in hot water.

### BRIEF DESCRIPTION OF THE DRAWINGS

FIG. 1 are photographs each showing the width of growth inhibition zone (clear zone) in Example 2 of this invention in which MRSA (FIG. 1-A), wild-type Staphylococcus aureus (FIG. 1-B) and Staphylococcus aureus 209P (FIG. 1-C) and enterohermorrhagic E. coli (O-157; FIG. 1-D).

FIG. 2 are photographs each showing the width of growth of inhibition zone (clear zone) in Example 3 of this invention in which MRSA(FIG. 2-A), wild-type Staphylococcus aureus (FIG. 2-B)and Staphylococcus aureus 209P (FIG. 2-C) and enterohermorrhagic E. coli(O-157; FIG. 2-D).

### DETAILED DESCRIPTION OF THE PREFERRED EMBODIMENTS OF THE INVENTION

The inventors of this application have researched and studied particularly tealeaves to provide an antimicrobial agent which is efficacious in treating or preventing infections with Staphylococccus, especially MRSA. Specifically, the inventors examined 35 kind of tealeaves whose origin is Japan, 15 kind of tealeaves whose origin is Taiwan, and 5 kind kof tealeaves whose origin is China to examine antimicrobial effects against a variety of Staphylococcus including MRSA. As a result of examinations, the inventors of this application found that two kind of tealeaves and/or tea stems, namely, “shan-lin-xi-gao-shan-cha” and “bai-hao-wu-long” both of whose origin is Taiwan are efficacious in inhibiting growth of MRSA and accomplished this invention.

Generally, tea is produced by nipping sprouts (young leaves) of tea-trees, which belongs to Fam. Theaceae and by processing the tealeaves according to a specified production process. Tea is mainly classified into unfermented tea (green tea), semi-fermented tea (oolong tea or “wu-long cha” and the like), and completely fermented tea (black tea) based on a degree of fermentation. For example, semi-fermented tea is mainly classified into “wu-long-cha” and “bao-zhong-cha” based on the difference of production process, kind of raw material, shape of tealeaves, etc.

Tea is not only favored as drinks of preference because of its sophisticated aroma and taste but also is known to have various physiological effects. For instance, catechin (tannin) which is contained in tealeaves at a large amount is known to have anti-cancer activity, anti-aging activity, cholesterol-reducing activity in blood, etc.

Recent study promotes usability of catechin in tea. For instance, there has been reported that decoction of a certain kind of green tea produced in Japan sterilizes toxic pathogenic enterohemohemorrhagic E. coli (O-157) and detoxicates vero toxin. The inventors of this application also examined catechin to find out whether catechin has antimicrobial effect against Staphylococcus, particularly MRSA. However, as a result of examination, there has been proved that catechin only, even if it has a high concentration, does not provide a desired antimicrobial effect.

Also, various kind of teas have been examined considering the content of catechin therein. However, there has not been found a significant correlation between catechin and antimicrobial effect against Staphylococcus. Taking an example of green tea produced in Japan, since green tea is drunk by pouring heated water of a relatively low temperature thereto, a large amount of catechin which is a component which causes a taster to feel bitterness is eluted in the tea. Further, there is another kind of tea in Japan, so-called “ho-ji-cha”, which is obtained by roasting decocted green tea according to a specified process. Catechin in the roasted tea reduces by heating in the roasting process. Further, semi-fermented tea such as oolong tea and “bao-zhong-cha” is drunk by pouring boiling water to steep for 3 to 5 minutes. Accordingly, the amount of catechin in the semi-fermented tea reduced less than half of catechin in green tea. Thus, a variety of teas having different contents of catechin have been examined with respect to antimicrobial effect against MRSA. As a result of examination, it has been verified that tealeaves and/or tea stems of “bai-hao-wu-long” produced in Taiwan, which is a kind of oolong tea and is considered to contain less amount of catechin compared to green tea in Japan as mentioned above, provides excellent antimicrobial effect against MRSA. On the other hand, tealeaves of “dong-ding-wu-long” produced in Taiwan failed to provide antimicrobial effect against MRSA. In addition, “shan-lin-xi-gao-shan-cha” produced in Taiwan is drunk in the same manner as oolong tea. It has been verified that tealeaves and/or tea stems of “shan-lin-xi-gao-shan-cha” produced in Taiwan also provides excellent antimicrobial effect against MRSA. The above facts clarify that the antimicrobial effect by the antimicrobial agent of this invention is not necessarily caused by catechin independently.

In fact, 15 g of tealeaves of “shan-lin-xi-gao-shan-cha” was poured into hot water (650 ml) at 90° C. to stand at room temperature for 30 seconds. The resulting soluble extract contains 0.01% (0.03 g/100 g) tannin (as tannic acid) using FOLIN-DENIS methods. In a similar method, soluble extract of “bai-hao-wu-long” is not detected tannin (as tannic acid; detection limit: 0.01 g/100 g).

Reasons as to why the above two tealeaves and/or tea stems exhibit excellent antimicrobial effect have not been elucidated in detail. However, considering the fact that catechin by itself which has been known as an active ingredient in tealeaves does not provide antimicrobial effect against Staphylococcus including MRSA based on the results of the above basic experiments, it is considered that a desired antimicrobial effect of the antimicrobial agent of this invention is obtained by combination of various factors such as soils, climate, cultivating conditions for producing the above two kind of tealeaves in Taiwan, as well as active ingredients therein.

Now, tealeaves and/or tea stems used in this invention are described

“Bai-hao-wu-long” is a semi-fermented tea using mainly “qingxindaiyou”, “tai-cha-17” and “bai-mao-hou”. “Bai-hao-wu-long” is particularly produced from tea buds, having high fermentation degree of about 60 to 70%. It is producible exclusively in Taiwan (manufactured by Xin Hua Cha Hang), and is known as a kind of oolong tea which is close to black tea in the degree of fermentation.

Preparation process:

(1) exposing tealeaves to weak sunlight for a long period until the tealeaves shrink to such an extent that tea buds exhibit somewhat whitish color and the surface of tea stems show wrinkle-like shrinkage. Thereupon, the shrunk tealeaves are moved inside a teahouse, and stirred until aroma comes out.

(2) roasting the thus prepared tealeaves at a low temperature, and covering the roasted tealeaves with a wet cloth for rubbing.

(3) rubbing the tealeaves gently without crushing the tealeaves into pieces.

(4) drying in hot air of about 90° C.

“Shan-lin-xi-gao-shan-cha is a semi-fermented tea using mainly “half-spheric-type oolong tea” (for example, qingxin-oolong tea, etc.) and has fermentation of about 15 to 30% Tealeaves and/or tea stems of “shan-lin-xi-gao-shan-cha” is cultivated in the valley of cypress trees in the Central Mountains in Taiwan, and is known to be a high-grade tea having sophisticated aroma (manufactured by Xin Hua Cha Hang).

Property: tealeaves exhibit bluish green color because they are grown under an alpine climate, low bitterness with excellent astringency and aroma

Preparation process, same as generally-known wu-long (oolong) tea; exposing to sunlight→moving in a teahouse for stir and fermentation→roasting→heating→rubbing→drying

The antimicrobial agent of this invention contains, as an active ingredient, the extract of the aforementioned tealeaves and/or tea stems. The extract is obtained by extraction in hot water. It is recommended to adjust the concentration of the extract at about 2 to 15%, preferably at 2 to 10% in order to obtain a desired antimicrobial effect. As will be clear from the examples which will be described later, the higher the concentration of the extract is, the higher the antimicrobial effect is secured. Generally, however, it is recommendable to prepare the extract by pouring hot water of about 100 cc with respect to the tealeaves and/or tea stems of about 2 to 10 g in view of easy dosage. For the most popular way, in the case where the extract is used in the form of teabag, a preferable dosage is such that a teabag containing 2.5 g or 5.0 g of tealeaves and/or tea stems is prepared and hot water of about 100 cc in a temperature from 65 to 95° C. is poured to obtain the extract. It is recommended to dose the thus prepared soluble extract having a certain concentration in the amount of about 1.2 to 2.0 cc/kg each time for about 3 to 4 times a day as the amount of the effective ingredient per adult for oral.

According to this invention, the extract of tealeaves and/or tea stems of “shan-lin-xi-gao-shan-cha” and “bai-hao-wu-long” produced in Taiwan may be used alone or in combination. In the case where the two kind of tealeaves and/or tea stems are used in combination, it is preferable to use them substantially at an equivalent amount so that the total content thereof lies within the aforementioned range. The content of the extract should be dosed appropriately, depending on the extent of the condition of the patient, the age and the like.

The antimicrobial agent of this invention is applicable to Staphylococcus, specifically, bacteria belonging to genus Staphylococcus such as Staphylococcus aureus (S. aureus) including MRSA, Staphylococcus epidermidis (S. epidermidis), and Staphylococcus saprophyticus (S. saprophyticus). Particularly, the inventive antimicrobial agent exhibits excellent antimicrobial effect against MRSA.

For the formulation of the present antimicrobial agents, it may be used appropriately flavorings, sweetening agents, preservatives, stabilizers, vitamin C, vitamin E, vitamin K, folic acid, etc.

Hereinafter, the invention is described in detail with reference to examples. It should be noted that the invention is not limited by the examples at all and that any modification and alteration of this invention which does not depart from the spirit of essential characteristics of the aforementioned and below-mentioned description are construed to be embraced in the technical range of this invention.

### EXAMPLES

### Example 1

Sensitivity test against MRSA using a variety of kind of tealeaves produced in Taiwan and China

The following experiments were performed by using various kind of tealeaves produced in Taiwan as shown in Table 1 and various kind of tealeaves produced in China as shown in Table 2.

First, Methicillin-Resistant Staphylococcus aureus (MRSA) was incubated in neutrient agar medium for proliferation cultivation to examine drug sensitivity in accordance with Halo-test, that is, disc agar diffusing capacity test. Specifically, extracts respectively having the concentrations as shown in Tables 1 and 2 were absorbed in paper discs of 8 mm diameter (manufactured by Showa Disc Kabushiki Kaisha). MRSA proliferation cultured was smeared on each medium. The above discs were placed on each medium at three positions with triangle shape. Then, the mediums were incubated in an incubator at 37° C. for 24 to 48 hours. The width (mm) of growth inhibition zone (clear zone) on the mediums was measured, and the result of measurements is shown in Table 1.

TABLE 1

| | Extract concentration % | | | | | |
|---|---|---|---|---|---|---|
| Kind of tealeaves | 2.5 | 5.0 | 7.5 | 10.0 | 12.5 | 15.0 |
| dong-ding-wu- | — | — | — | — | 1.5 | 1.5 |
| long cha | | | | | | |
| an-xi-tie-guan-yin | — | — | — | — | — | — |
| mu-shan-tie-guan- | — | — | — | — | — | — |
| yin | | | | | | |
| wang-shan-cha | — | — | — | — | — | — |
| Shi-gu-wu-long | — | — | — | — | — | 1.0 |
| a-li-shan-cha | — | — | — | — | — | — |
| Gao-shan-cha | — | — | — | — | 1.0 | 2.0 |
| Jin-xuan-cha | — | — | — | — | — | — |
| bai-hao-wu-long | 10.5 | 12.5 | 14.0 | 15.0 | 16.0 | 17.0 |
| shan-lin-xi-gao- | 9.5 | 11.0 | 13.5 | 15.5 | 17.5 | 18.5 |
| shan-cha | | | | | | |

TABLE 2

| | Extract concentration (%) | | | | | |
|---|---|---|---|---|---|---|
| Kind of tealeaves | 2.5 | 5.0 | 7.5 | 10.0 | 12.5 | 15.0 |
| Wu-yi-tan-cha | — | — | — | — | 1.0 | 2.0 |
| long-jing-cha | — | — | — | — | 1.0 | 1.0 |
| bi-luo-chun | — | — | — | — | — | — |
| Huang-shan-mao-feng | — | — | — | — | — | — |
| jing-shan-xiang-cha | — | — | — | — | — | 1.0 |

As a result of these experiments, it was verified that only the extract of the tealeaves of “bai-hao-wu-long” and “shan-lin-xi-gao-shan-cha” among the variety of kind of tealeaves produced in Taiwan and China shown in Tables 1 and 2 exhibited excellent antimicrobial effect against MRSA even at such a low concentration as 2.5%.

On the other hand, almost no antimicrobial effect has been found with respect to the extracts of the tealeaves other than the aforementioned two kind. Only in the case where the extracts of the other kind of tealeaves had a high concentration ranging from 12-5 to 15.0%, these extracts showed a very weak antimicrobial effect.

Although not shown, experiments using bacteria which belongs to genus Staphylococcus other than MRSA were performed, and it was verified that similar results as the results in the aforementioned Example 1 were obtained.

### Example 2

Sensitivity test against various kind of bacteria using the extract of “shan-lin-xi-gao-shan-cha”

(1) Test Bacteria

The following thirteen kind of bacteria were used for the experiment: Staphylococcus such as Methicillin-Resistant Staphylococcus aureus (MRSA), Staphylococcus aureus (wild-type Staphylococcus aureus and Staphylococcus aureus 209P), Staphylococcus epidermidis and Staphylococcus haemolyticus, Staphylococcus saprophyticus; Streptococcus (Streptococcus pneumoniae and Streptococcus agalactiae); Enterococcus faecalis; Escehrichia [enterohemohemorrhagic E. coli (O157); and Salmonellas (Salmonella enteritidis and Salmonella thyphimurium).

(2) Test Method

Test method of Example 2 is conducted in a similar to that of Example 1 except selecting an optimal cultivation depending on the kind of bacteria. Sensitivity test against each bacteria using the extract of tealeaves of “shan-lin-xi-gao-shan-cha” was measured in the same manner as Example 1. The result of the measurements is shown in Table 3.

TABLE 3

| | Extract concentration (%) | | | | | | |
|---|---|---|---|---|---|---|---|
| Kind of bacteria | 1.25 | 2.5 | 5.0 | 7.5 | 10.0 | 12.5 | 15.0 |
| MRSA | 4.5 | 6.5 | 9.0 | 10.5 | 11.5 | 12.5 | 13.5 |
| wild-type Staphylococcus aureus | 1.5 | 3.0 | 5.5 | 7.5 | 8.5 | 10.0 | 12.0 |
| Staphylococcus aureus 209P | 2.0 | 4.0 | 6.5 | 9.5 | 10.0 | 10.5 | 11.0 |
| Staphylococcus epidermidis | 0 | 0 | 0 | 1.0 | 1.5 | 2.0 | 2.0 |
| Staphylococcus haemolyticus | 0 | 0 | 0 | 0 | 1.0 | 1.5 | 1.5 |
| Staphylococcus saprophyticus | 0 | 0 | 1.0 | 1.5 | 2.0 | 2.5 | 2.5 |
| Staphylococcus pneumoniae | 0 | 0 | 1.5 | 2.0 | 4.0 | 4.5 | 5.0 |
| Staphylococcus agalactiae | 0 | 0 | 0 | 1.5 | 1.5 | 2.0 | 2.5 |
| Enterococcus faecalis | 0 | 0 | 0 | 1.0 | 2.0 | 2.5 | 3.0 |
| Escherichia coli | 0 | 0 | 0 | 0.5 | 1.0 | 1.5 | 2.0 |
| enterohermorrhagic E. coli, O-157 | 0 | 0 | 0.8 | 2.5 | 3.0 | 3.0 | 3.0 |
| Salmonella enteritidis | 0 | 0 | 0 | 0 | 1.0 | 1.5 | 2.0 |
| Salmonella typhimurium | 0 | 0 | 0 | 1.5 | 2.5 | 3.0 | 3.0 |

As is clear from Table 3, it was found that “shan-lin-xi-gao-shan-cha” extract has excellent antimicrobial effect against Staphylococcus, particularly, MRSA. The width of growth inhibition zone (clear zone) with MRSA was remarkably larger than that of Staphylococcus aureus 209P and wild type Staphylococcus aureus.

On the other hand, almost no antimicrobial effect of “shan-lin-xi-gao-shan-cha” was found against the bacteria other than Staphylococcus. A very weak antimicrobial effect was found only in the case where the concentration of extract was increased.

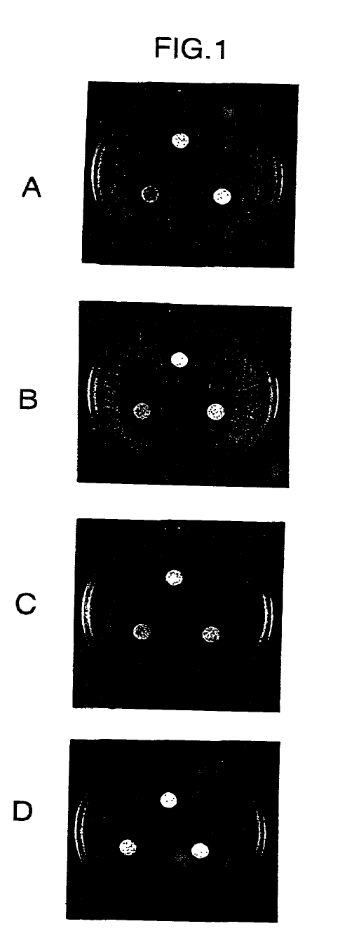

FIG. 1 are photographs each showing the width of growth inhibition zone (clear zone) in Example 2 in which MRSA (FIG. 1-A), wild-type Staphylococcus aureus (FIG. 1-B) and Staphylococcus aureus 209P(FIG. 1-C) and enterohermorrhagic E. coli (O-157; FIG. 1-D) were used.

### Example 3

Sensitivity test against various kind of bacteria using “bai-hao-wu-long” tea

Sensitivity test was performed in the similar manner as Example 2 except that “bai-hao-wu-long” tealeaves was used in place of “shan-lin-xi-gao-shan-cha” tealeaves. The results of experiments are shown in Table 4.

TABLE 4

| | Extract concentration on disc (%) | | | | | | |
|---|---|---|---|---|---|---|---|
| Kind of bacteria | 1.25 | 2.5 | 5.0 | 7.5 | 10.0 | 12.5 | 15.0 |
| MRSA | 2.5 | 4.0 | 6.8 | 7.0 | 8.0 | 9.0 | 10.5 |
| wild-type Staphylococcus aureus | 1.5 | 2.7 | 2.7 | 3.0 | 3.5 | 4.0 | 4.5 |
| Staphylococcus aureus 209P | 2.0 | 2.5 | 3.5 | 5.0 | 5.5 | 6.0 | 6.5 |
| Staphylococcus epidermidis | 0 | 0 | 0 | 1.0 | 1.5 | 2.0 | 2.0 |
| Staphylococcus haemolyticus | 0 | 0 | 0 | 0 | 0.5 | 1.0 | 1.0 |
| Staphylococcus saprophyticus | 0 | 0 | 0 | 0 | 1.0 | 1.5 | 1.5 |
| Staphylococcus pneumomiae | 0 | 0 | 1.0 | 1.5 | 2.0 | 2.5 | 3.0 |
| Staphylococcus agalactiae | 0 | 0 | 0 | 1.5 | 1.5 | 2.0 | 2.5 |
| Enterococcus faecallis | 0 | 0 | 0 | 1.0 | 1.5 | 2.0 | 2.5 |
| Escherichia coli | 0 | 1.0 | 0 | 0 | 0 | 0 | 0 |
| enterohermorrhagic E. coli, O-157 | 0 | 1.0 | 1.5 | 0 | 0 | 0 | 0 |
| Salmonella enteritidis | 0 | 0 | 0 | 0 | 0 | 0 | 0 |
| Salmonella typhimurium | 0 | 0 | 0 | 0 | 0 | 0 | 0 |

As is clear from Table 4, “bai-hao-wu-long” tealeaves exhibited excellent antimicrobial effect against Staphylococcus as with the case of Example 2 in which “shan-lin-xi-gao-shan-cha” tealeaves was used. It was verified that the extract of “bai-hao-wu-long” tealeaves has excellent antimicrobial effect against MRSA as with the case of Example 3 in which the extract of “shan-lin-xi-gao-shan-cha” tealeaves was used.

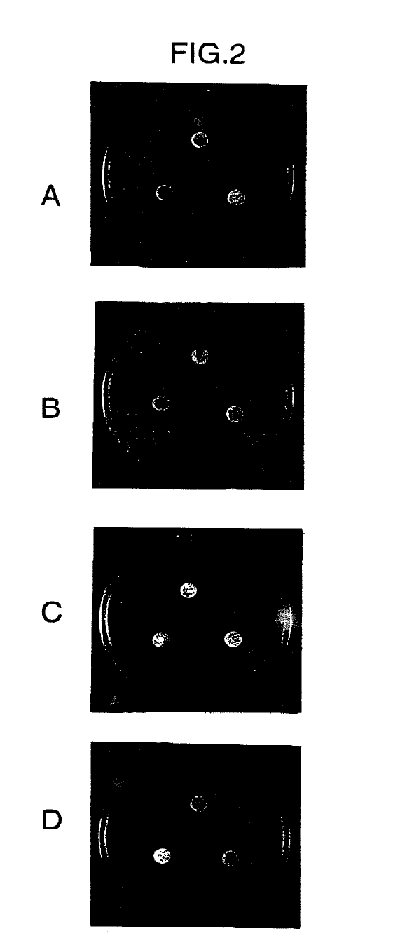

FIG. 2 are photographs each showing the width of growth of inhibition zone (clear zone) in Example 3 in which MRSA(FIG. 2-A), wild-type Staphylococcus aureus (FIG. 2-B)and Staphylococcus aureus 209P (FIG. 2-C) and enterohermorrhagic E. coli (O-157; FIG. 2-D) were used.

### Example 4

Test of verifying sterilizing effect (in vitro)

The following in vitro test; was performed to verify the sterilizing effect against Staphylococcus using the extract of the present invention.

Specifically, 5% of each extract solution (5 cc) of “shan-lin-xi-gao-shan-cha” and “bai-hao-wu-long” was added in a test tube each containing 5 cc of three kind of MRSA shown in Table 3. Then, the test tubes were incubated in an incubator at 37° C. for 24 hours, and then were centrifugalized to give a residue. The resulting residue was smeared on Staphylococcus No. 110 mediums (manufactured by Nissui & Co., registered trademark). Then, the mediums were cultured in the incubator for 24 hours. The evaluation of the sterilizing effect was conducted as to the presence or absence of colony formation. The evaluation result “-” indicates that no colony formation was founded.

The results of the evaluation are also shown in Table 5.

TABLE 5

| | Diluting magnification of suspension containing bacteria | | | | | | | |
|---|---|---|---|---|---|---|---|---|
| | 100 | | 1,000 | | 5,000 | | 10,000 | |
| Kind of tealeaves | A | B | A | B | A | B | A | B |
| Extract concentration (%) | 5.0 | 5.0 | 5.0 | 5.0 | 5.0 | 5.0 | 5.0 | 5.0 |
| Used amount of suspension (ml) | 5.0 | 5.0 | 5.0 | 5.0 | 5.0 | 5.0 | 5.0 | 5.0 |
| Added amount of extract (ml) | 5.0 | 5.0 | 5.0 | 5.0 | 5.0 | 5.0 | 5.0 | 5.0 |
| Kind of bacteria | | | | | | | | |
| MRSA (derived from human being) | (—) | (—) | (—) | (—) | (—) | (—) | (—) | (—) |
| MRSA (derived from dairy cattle) | (—) | (—) | (—) | (—) | (—) | (—) | (—) | (—) |
| MRSA (derived from dog) | (—) | (—) | (—) | (—) | (—) | (—) | (—) | (—) |

Note:
A: shan-lin-xi-gao-shan-cha
B: bai-hao-wu-long

As is clear from Table 5, in the case where the extract of the present invention (each tealeaves of “shan-lin-xi-gao-shan-cha” and “bai-hao-wu-long”) was used, colony formation was not founded. The results of these experiments verifies excellent sterilizing effect against MRSA by the present invention.

### Example 5

Test of verifying sterilizing effect (in vivo)

The following in vivo test was performed by using rats to further verify the sterilizing effect of the antimicrobial agent of this invention in addition to the aforementioned in vitro test.

Specifically, 24 healthy adult male SD strain, SPF rats were divided into 6 groups (4 rats per group). 5 cc of suspensions respectively containing the three kind of MRSA shown in Table 6 were dosed per orally of each rat by a probe for rats. Upon lapse of 30 minutes after dosing, each 5 cc of 5% extract solution from the respective kind of tealeaves shown in Table 6 was dosed per orally of each rat in the similar manner as dosing the suspension containing MRSA. Upon lapse of 24 to 48 hours, dip from feces of the rats was smeared on Staphylococcus No. 110 mediums to incubate in an incubator of 37° C. for 24 to 48 hours. An identification of Staphylococcus was performed by Gram's staining method and microscopic observation. The results of the observation are also shown in Table 6. It should be noted that no Staphylococcus found in the feces of the tested rats before the aforementioned experiments were performed.

TABLE 6

| | | | Body | Dose amount | Dose amount | Detection | Detection |
|---|---|---|---|---|---|---|---|
| Kind of | Tea- | | Weight | of suspension | of extract | of bacteria | of bacteria |
| bacteria | leaves | Animal | (g) | (ml) | (ml) | after 24 hours | after 48 hours |
| MRSA (derived | A | male rat | 355 | 5.0 | 5.0 | — | — |
| from human) | B | male rat | 360 | 5.0 | 5.0 | — | — |
| MRSA (derived | A | male rat | 357 | 5.0 | 5.0 | — | — |
| from dairy cattle) | B | male rat | 355 | 5.0 | 5.0 | — | — |
| MRSA (derived | A | male rat | 362 | 5.0 | 5.0 | — | — |
| from dog) | B | male rat | 358 | 5.0 | 5.0 | — | — |

Note:
A: shan-lin-xi-gao-shan-cha
B: bai-hao-wu-long

As is clear from Table 6, colony formation was not found on all the mediums. This result verifies sterilizing effect against MRSA by the extract of this invention.

As mentioned above, the antimicrobial agent of this invention exhibits a remarkably efficacious antimicrobial effect against Staphylococcus, particularly MRSA. In addition, this invention has the great advantage of no side-effects and easiness to dose for patients, because of using the aforementioned tealeaves and/or tea stems.

This application is based on patent application No. 2000-348728 filed in Japan, the contents of which are hereby incorporated by references.

As this invention may be embodied in several forms without departing from the spirit of essential characteristics thereof, the present embodiment is therefore illustrative and not restrictive, since the scope of the invention is defined by the appended claims rather than by the description preceding them, and all changes that fall within metes and bounds of the claims, or equivalence of such metes and bounds are therefore intended to embraced by the claims.

What is claimed is:

1. A method for treating and/or preventing infections caused by Methicillin-Resistant Staphylococcus aureus in human and non-human animals comprising administering an effective amount of an antimicrobial agent consisting essentially of a soluble extract of tealeaves and/or tea stems of “shan-lin-xi-gao shan-cha” and/or “bai-hao-wu-long” whose place of origin is Taiwan, wherein the extract is obtained by extraction in hot water.
